(19) United States
(12) Patent Application Publication
(10) Pub. No.: US 2004/0193087 A1
(43) Pub. Date: Sep. 30, 2004

(54) Wound dressing product with removal aid and a method of manufacturing same

(76) Inventors: Sascha Albus, Melsbach (DE); Joachim Thewalt, Melsbach (DE); Heike Grothaus, Kurtscheid (DE); Martin Kaiser, Neuwied (DE); Andreas Hahn, Neuwied (DE); Gabriel Slupik, Woldert (DE)

Correspondence Address: Friedrich Kueffner, 317 Madison Avenue, Suite 910, New York, NY, 10017, US

(21) Appl. No.: 10/803,801

(22) Filed: Mar. 18, 2004

(30) Foreign Application Priority Data
Mar. 20, 2003 (DE) 103 12 452.7

(51) Int. Cl. A61F 13/00

(52) U.S. Cl. 602/42

(57) ABSTRACT

A wound dressing product has a web-shaped wound dressing element having an adhesive surface. A cover arrangement is provided for covering the adhesive surface, wherein the cover arrangement has at least two cover elements. At least one removal aid is connected to at least one of the cover elements for facilitating removal of the cover elements from the wound dressing element. The cover elements are separated from one another by a partition line. The removal aid that is connected to the associated cover element extends in a direction away from the partition line and away from the opposed cover element and overlaps the associated cover element.

### BACKGROUND OF THE INVENTION

1. Field of the Invention

The invention relates to a wound dressing product comprising a strip-shaped or web-shaped wound dressing element that is provided with a sticky adhesive surface and further comprising a cover arrangement, for covering the adhesive area, having at least two cover elements. At least one of the cover elements has correlated therewith a removal aid for facilitating removal of the cover element from the strip-shaped or web-shaped wound dressing element. The invention further relates to a method for manufacturing such wound dressing products.

2. Description of the Related Art

Such wound dressing products are manufactured in the form of rolls with different widths, for example, 107 mm and 157 mm. They are used in prophylactic applications (inter alia, decubitus) and for fixation and covering in connection with the treatment of wounds. For this purpose, the required product lengths are cut to length from the roll. Subsequently, the cover element are removed from the adhesive surface by using the removal aid and, finally, the wound dressing element is applied with its adhesive surface. In conventional wound dressing products of the aforementioned kind, the covering arrangement comprises two overlapping cover elements each having a removal aid in the form of an end strip of the cover element that is folded onto itself. Such wound dressing products are not only difficult to apply but are also problematic with regard to their manufacture.

### SUMMARY OF THE INVENTION

It is an object of the present invention, in view of the aforementioned problems of the prior art, to provide a wound dressing product of the aforementioned kind that can be produced in a simple way and enables, despite its simple manufacture, an easy application. Moreover, it is an object of the present invention to provide a suitable method for manufacturing such wound dressing products.

In accordance with the present invention, this is achieved in that, as a further development of known wound dressing products, the product according to the invention is characterized essentially in that the cover elements are separated from one another by a partition line and in that at least one removal aid extends, starting at the partition line, in a direction away from the neighboring cover element and overlaps the cover element it is associated with.

According to the invention, cover elements are thus used that directly abut one another without overlapping one another. In this way, the application of the wound dressing product or of the wound dressing element is facilitated because the cover elements can be removed from the adhesive surface of the wound dressing elements without having to deal with mutual overlap of the cover elements. Moreover, the manufacture of the wound dressing products according to the invention is particularly simple because no overlap of the cover elements must be effected during the course of the manufacturing process. In this connection, the invention is based on the realization that a complete covering of the adhesive surface, assumed to be necessary according to the prior art and effected by the covering arrangement realized by the mutual overlap of the cover elements, is not required in many cases. This is particularly true in cases where the wound dressing product is used only for fixation and covering in the treatment of wounds. With regard to a simple manufacture and handling, a narrow exposed strip of the adhesive surface in the area of the partition line is acceptable without this causing a significant impairment of the application properties of the wound dressing product.

In regard to a further simplification of the application of the wound dressing products according to the invention, it was found to be advantageous when each one of the cover elements, separated from one another by the partition line, has associated therewith a removal aid that extends, starting at the partition line, in a direction away from the neighboring cover element. The latter embodiment of the invention can be produced particularly simply when the removal aids, associated with the cover elements that are. separated from one another by the partition line, are provided with removal strips that are separated from one another by the partition line and fastened to the cover elements by adhesive strips: the adhesive strips are separated from one another by the partition line and extend approximately parallel to the partition line. Such wound dressing products can be manufactured particularly simply without requiring a folding process for realizing the removal aids in that an adhesive surface of a strip-shaped wound dressing element is covered with a cover strip; subsequently, a web-shaped removal aid arrangement is attached by means of two approximately parallel extending adhesive strips on the cover strip; and, subsequently, a partition line is formed between these adhesive strips, wherein the partition line penetrates the cover strip and the removal aid arrangement in order to form in this way two separate cover elements with removal strips fastened thereto.

A further simplification of application of the wound dressing products according to the invention can be achieved when at least one removal aid is visually distinguishable, in particular, by color, and/or haptically distinguishable from the associated removal aids or elements. In this case, the removal aid can be recognized quickly after cutting a strip of a suitable length from the remainder of the rolled-up web-shaped wound dressing product and can be gripped safely for the purpose of applying the wound dressing element. In this connection, the removal aid can be made of a different material. In particular, it is proposed to realize the removal aid in the form of conventional (colored) paper strips and to realize the cover elements in the form of silicone paper.

An accidental removal of the removal elements in the area of the lateral edges of the wound dressing product can be prevented when the covering arrangement surrounds or encloses the web-shaped element at least in the area of one of the lateral edges of the wound dressing element and, preferably, a reinforcement element is provided on at least one enclosed peripheral edge of the web-shaped wound dressing element. In this connection, the cover elements can be connected to one another by means of a support film that extends across the boundary surface of the wound dressing element that is facing away from the adhesive surface. As an alternative or additionally, the support film can be connected by means of separate reinforcement strips to the cover element. In other words, this means that the support film can be produced as a unitary part of the cover elements or can be a part separate from the cover element.

### BRIEF DESCRIPTION OF THE DRAWING

In the drawing:

FIG. 1 is a plan view onto the wound dressing product according to the invention;

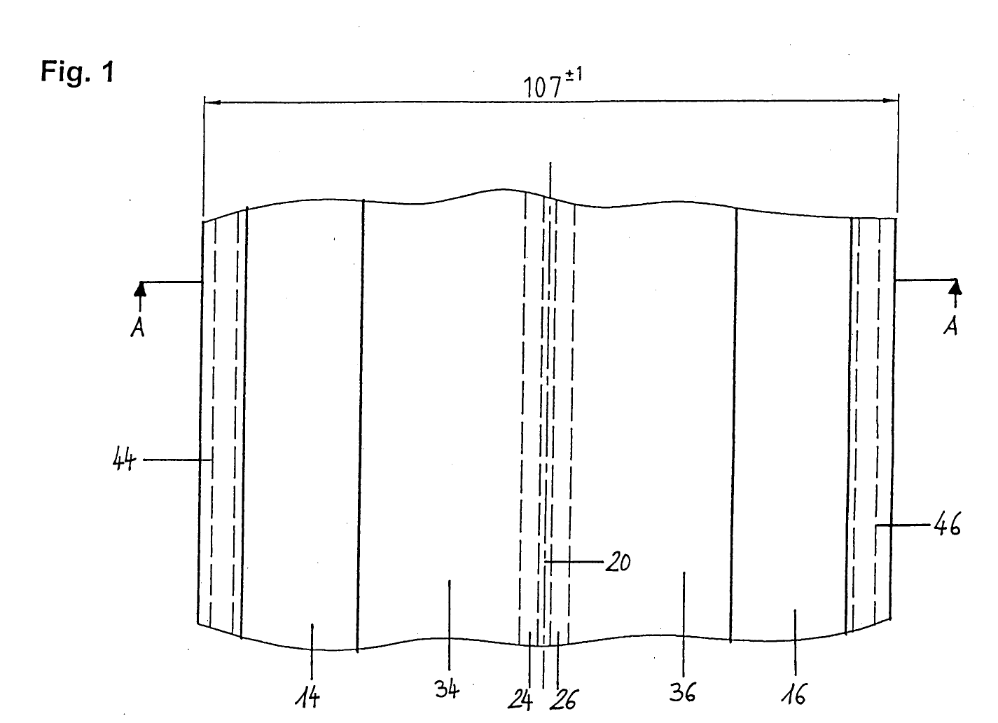

FIG. 2 is a section view of the wound dressing product illustrated in FIG. 1 along the section line II-II of FIG. 1.

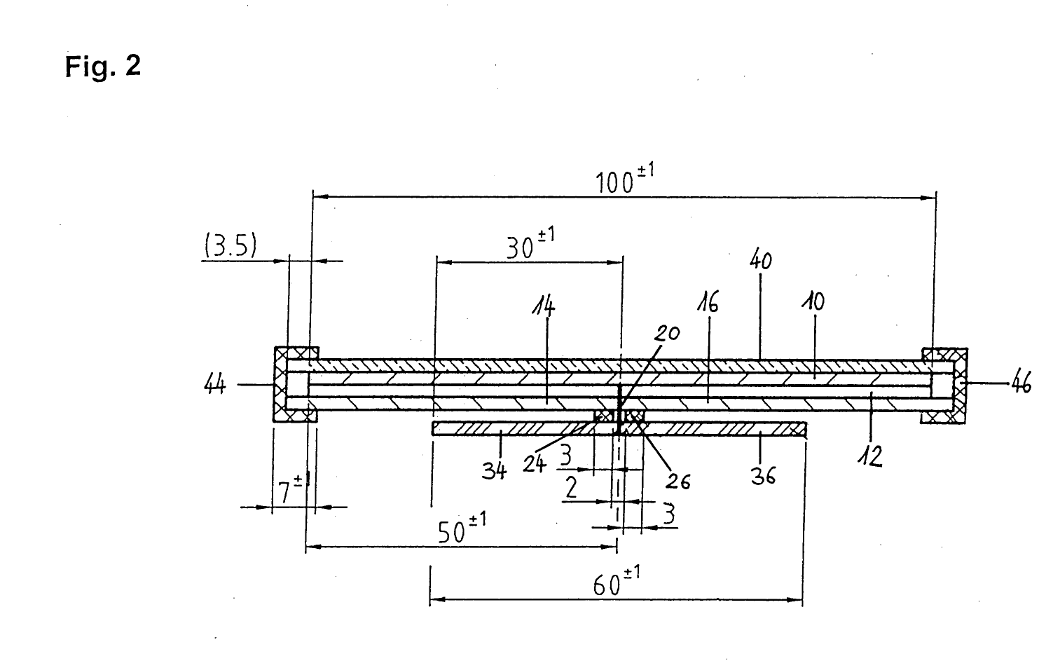

### DESCRIPTION OF THE PREFERRED EMBODIMENTS

The wound dressing product illustrated in the drawing comprises a web-shaped or strip-shaped wound dressing element 10 realized in the form of a PU (poly urethane) film. On an adhesive surface that is formed by an adhesive layer 12 of the wound dressing element 10 two cover elements 14 and 16 forming a cover arrangement are arranged. The cover elements 14 and 16 are separated from one another by a partition line 20 and have no mutual overlap. In the area of the partition line 20, the facing edges of the cover elements 14 and 16 abut one another. On the cover element 14, a removal strip 34 is attached by means of an adhesive strip 24. On the cover element 16, a removal strip 36 is attached by means of an adhesive strip 26. The removal strip 34, 36 forms the removal aid, respectively. The cover elements 14 and 16 are comprised of silicone paper; the removal strips 34 and 36 are comprised of colored paper. The cover elements 14 and 16 are connected to a support film 40 by means of reinforcement elements 44, 46 enclosing or surrounding the lateral edges of the wound dressing element 10. The support film 40 covers a boundary surface of the wound dressing element 10 facing away from the adhesive surface 12.

For manufacturing the wound dressing product illustrated in the drawing, a cover strip is fastened by means of the adhesive layer 12 on the wound dressing element 10. Subsequently, a removal aid arrangement in the form of a strip-shaped removal aid member is fastened by means of two parallel extending adhesive strips 24 and 26 on the boundary surface of the cover strip facing away from the adhesive surface 12. Subsequently, the removal aid member and the cover strip are cut by stamping along a partition line extending between the adhesive strips 24 and 26. When doing so, attention is paid that the stamping means does not cut through the wound dressing element 10. In this way, the cover elements 14 and 16 as well as the removal strips 34 and 36 are formed from the cover strip and the removal aid member, respectively. In this connection, the attachment of the removal aid member on the cover strip by means of the adhesive strips 24, 26 can be realized before or after application of the cover strip on the adhesive surface of the wound dressing element.

The invention is not limited to the embodiment illustrated in the drawing. It is also considered to cover the adhesive surface of the wound dressing element by a cover arrangement of three or more cover elements. Moreover, the support film 40 can be omitted. It is also proposed to fasten the removal strips on another location on the cover elements. Moreover, the product according to the invention can be produced in any desired length and width.

While specific embodiments of the invention have been shown and described in detail to illustrate the inventive principles, it will be understood that the invention may be embodied otherwise without departing from such principles.

What is claimed is:

1. A wound dressing product comprising:
a web-shaped wound dressing element having an adhesive surface;
a cover arrangement for covering the adhesive surface, wherein the cover arrangement has at least two cover elements;
wherein the at least two cover elements are separated from one another by a partition line;
at least one removal aid connected to at least one of the at least two cover elements for facilitating removal of the at least two cover elements from the wound dressing element;
wherein the at least one removal aid connected to the at least one of the at least two cover elements extends in a direction away from the partition line and overlaps the at least one of the at least two cover elements to which the at least one removal aid is connected.

2. The wound dressing product according to claim 1, wherein the at least one removal aid comprises a removal strip and an adhesive strip extending approximately parallel to the partition line and fastening the removal strip to the at least one of the at least two cover elements.

3. The wound dressing product according to claim 1, wherein the at least two cover elements each have one of the at least one removal aid.

4. The wound dressing product according to claim 3, wherein the removal aids each comprise a removal strip and an adhesive strip extending essentially parallel to the partition line, wherein the removal strips are fastened by the adhesive strips to the cover elements, wherein the adhesive strips and the removal strips are separated from one another by the partition line.

5. The wound dressing product according to claim 1, wherein the at least one removal aid is distinguishable by at least one of visual appearance and haptic properties from the at least two cover elements.

6. The wound dressing product according to claim 5, wherein the at least one removal aid is distinguishable by color from the at least two cover elements.

7. The wound dressing product according to claim 5, wherein the at least one removal aid is comprised of a material different from a material of the at least two cover elements.

8. The wound dressing product according to claim 1, wherein the cover arrangement encloses the wound dressing. element.

9. The wound dressing product according to claim 1, wherein the cover arrangement comprises at least one reinforcement element provided on a lateral edge of the wound dressing element.

10. A method for manufacturing a wound dressing product according to claim 1, comprising the steps of:
covering an adhesive surface of the wound dressing element by a cover strip;
attaching a web-shaped removal aid arrangement to a boundary surface of the cover strip facing away from the wound dressing element;
subsequently, forming a partition line penetrating the cover strip and the removal aid arrangement for producing two separate cover elements each having a removal strip fastened thereto.

11. The method according to claim 10, wherein the partition line is formed by stamping.

12. The method according to claim 10, wherein the removal aid arrangement is attached by two substantially parallel extending adhesive strips on the cover strip and wherein the partition line is formed between the two adhesive strips.
